(19) United States
(12) Patent Application Publication
(10) Pub. No.: US 2003/0134410 A1
(43) Pub. Date: Jul. 17, 2003

(54) Compositions and methods for performing biological reactions

(76) Inventors: Robin M. Silva, San Francisco, CA (US); George W. Hawkins, Gilbert, AZ (US)

Correspondence Address: DORSEY & WHITNEY LLP, INTELLECTUAL PROPERTY DEPARTMENT, 4 EMBARCADERO CENTER, SUITE 3400, SAN FRANCISCO, CA, 94111, US

(21) Appl. No.: 10/149,660

(22) PCT Filed: Dec. 15, 2000
(86) PCT No.: PCT/US00/34145

(51) Int. Cl. C12M 1/34; G06K 9/00

(52) U.S. Cl. 435/287.2; 382/128

(57) ABSTRACT

The present invention relates to an apparatus for performing biological reactions. Specifically, the invention relates to an apparatus for performing nucleic acid hybridization reactions on a substrate layer having a multiplicity of oligonucleotide binding sites disposed thereon, using substrates with flexible covers.

This application is a continuing application of U.S. Ser. Nos. 09/605,766, filed Jun. 28, 2000 and 09/464,490, filed Dec. 15, 1999.

### FIELD OF THE INVENTION

The present invention relates to an apparatus for performing biological reactions. Specifically, the invention relates to an apparatus for performing nucleic acid hybridization reactions on a substrate layer having a multiplicity of oligonucleotide binding sites disposed thereon, using substrates with flexible covers.

### BACKGROUND OF THE INVENTION

Recent advances in molecular biology have provided the opportunity to identify pathogens, diagnose disease states, and perform forensic determinations using gene sequences specific for the desired purpose. This explosion of genetic information has created a need for high-capacity assays and equipment for performing molecular biological assays, particularly nucleic acid hybridization assays. Most urgently, there is a need to miniaturize, automate, standardize and simplify such assays. This need stems from the fact that while these hybridization assays were originally developed in research laboratories working with purified products and performed by highly skilled individuals, adapting these procedures to clinical uses, such as diagnostics, forensics and other applications, has produced the need for equipment and methods that allow less-skilled operators to effectively perform the assays under higher capacity, less stringent assay conditions.

Nucleic acid hybridization assays are advantageously performed using probe array technology, which utilizes binding of target single-stranded DNA onto immobilized DNA (usually, oligonucleotide) probes. The detection limit of a nucleic acid hybridization assay is determined by the sensitivity of the detection device, and also by the amount of target nucleic acid available to be bound to probes, typically oligonucleotide probes, during hybridization.

Nucleic acid hybridization chambers are known in the prior art. U.S. Pat. No. 5,100,755 to Smyczek et al. discloses a hybridization chamber. U.S. Pat. No. 5,545,531 to Rava et al. discloses a hybridization plate comprising a multiplicity of oligonucleotide arrays. U.S. Pat. No. 5,360,741 to Hunnell discloses a gas heated hybridization chamber. U.S. Pat. No. 5,922,591 to Anderson et al. discloses a miniaturized hybridization chamber for use with oligonucleotide arrays. U.S. Pat. No. 5,945,334 to Besemer discloses oligonucleotide array packaging.

As currently employed, oligonucleotide array technology does not provide maximum hybridization efficiency. Existing nucleic acid hybridization assay equipment includes numerous components, each of which is a source of inefficiency and inaccuracy.

Hybridization using oligonucleotide arrays must be performed in a volume in which a small amount of target DNA or other nucleic acid can be efficiently annealed to the immobilized probes. For diagnostic assays, target DNA molecules are often obtained in minute (<picomol) quantities. In practice, it can take several (tens of) hours for hybridization to be substantially complete at the low target nucleic acid levels available for biological samples.

In addition, array hybridization is conventionally performed in a stationary hybridization chamber where active mixing is absent. Under these conditions, the probability that a particular target molecule will hybridize to a complementary oligonucleotide probe immobilized on a surface is determined by the concentration of the target, the diffusion rate of the target molecule and the statistics of interaction between the target and the complementary oligonucleotide. Consequently, a larger number of samples must be tested to obtain useful information, and this in turn leads to increased hybridization times and inefficiencies.

In addition, efficiency is increased when the amount of user manipulation is kept to a minimum. As currently performed, oligonucleotide array hybridization requires a great deal of operator attentiveness and manipulation, and the degree of skill required to perform the analysis is high. For example, hybridization is typically performed in an assay chamber, and then data collection and analysis are performed in a separate apparatus (such as a laser scanner or fluorescence microscope). This arrangement requires a substantial amount of handling by the user, and makes the assays both time-consuming and subject to user error.

It is also a limitation of current practice that array hybridizations are performed one array at a time, thereby forgoing the economies of parallel processing and data analysis.

Additional limitations, inefficiencies, and expenses arise from the structural characteristics of existing apparatus. Many existing apparatus are limited in the size of the substrate they can accommodate. Other apparatus are not disposable and therefore require extensive cleaning between runs in order to prevent sample contamination. Yet other apparatus are high mass and therefore not susceptible of the rapid heating and cooling necessary for efficient hybridization. Other apparatus require the use of expensive optics for analysis of the reaction products.

There remains a need in this art for an easy-to-use apparatus for performing biological reactions, particularly nucleic acid hybridization, that comprises a small reaction volume, where the fluid components can be actively mixed, that can be performed in parallel and that minimizes user intervention. There also remains a need for such an apparatus that is easy to manufacture in various sizes, that is disposable to minimize sample contamination, that allows for the use of low cost optical analytical equipment, and that is low mass to allow for rapid heating and cooling of the sample fluid. There also remains a need for methods for using such apparatus to increase hybridization efficiency, particularly relating to biochip arrays as understood in the art. This need is particularly striking, in view of the tremendous interest in biochip technology, the investment and substantial financial rewards generated by research into biochip technology, and the variety of products generated by such research.

### SUMMARY OF THE INVENTION

In accordance with the objects outlined above, the present invention provides apparatus for performing biological reactions, comprising a substrate that has a first and a second surface (usually opposite to each other, in the case of planar substrates). An array of biomolecules are positioned on the first surface, and a flexible layer affixed to the first surface of the substrate by an adhesive layer, wherein the adhesive layer is deposited on the first surface and forms a reaction volume. The substrate comprises at least a first port extending through the substrate from the first to the second surface, and the port has a first opening and a second opening. The first opening of the port is provided within the area bounded by the adhesive and covered by the flexible layer (e.g. the reaction volume) and the second opening of the port is provided on the second surface of the substrate. Alternatively, the port(s) may be in the flexible layer into the reaction volume.

Optionally, there may be a removable cover positioned over the second opening of the port such as a foil tape, as well as a layer of a water-soluble compound that is a solid at a first temperature and a liquid at a second, higher temperature, the layer being positioned between the first surface of the substrate and the flexible layer. Optionally, the flexible layer is a translucent plastic or gas permeable membrane.

The port can be in the shape of a truncated cone having a small-diameter end and a large diameter end, and wherein the small diameter end is provided in the area on the first substrate surface bounded by the adhesive layer (the reaction volume) and the large diameter end is provided on the second substrate surface. The walls of a port in some embodiments form an angle of less than 90° with the second substrate surface. Optionally, the port contains a sample fluid having a contact angle, and the angle formed between the second substrate surface and the port walls is less than or equal to the contact angle of the sample fluid.

A second port can be included; in this embodiment, the second port extends through the substrate from the first surface to the second surface thereof and having a first opening and a second opening, wherein the first opening of the outlet port is provided on the first substrate surface within the area bounded by the adhesive and covered by the flexible layer, and the second opening of the outlet port is provided on the second substrate surface and is covered by a removable cover. Alternatively, the second port is in the flexible layer as above.

Optionally, the apparatus of the invention can comprise a reflective layer positioned between the array and the first substrate surface, and/or a resistive heater; the latter can be a reflective layer positioned between the array and the first substrate surface.

Additionally, the apparatus can comprise a passivation layer such as parylene, or a scanner, wherein the scanner is in contact with the flexible layer at a position over the array. The scanner can be a light pipe and cover the entirety of the reaction volume.

In a further aspect, the apparatus further comprises a sample preparation chip in contact with the second substrate surface wherein the sample preparation chip has a port that is aligned with the first port of the apparatus.

In an additional aspect, the apparatus further comprises a roller, wherein the roller is in contact with the flexible layer at a position over the array; for example, it can move longitudinally across the reaction volume

In a further aspect, the apparatus can include a case having a lid and a base with a cavity, and a carriage comprising a scanner and a roller, wherein the carriage is provided in the cavity, wherein the substrate is removably positioned above the carriage such that the first substrate surface is in operative contact with the carriage.

In an additional aspect, the invention provides apparatus for performing biological reactions, comprising a glass microscope slide having a first surface and a second surface opposite thereto; an array of biomolecules positioned on the first surface of the slide, wherein each biomolecule within the array is anchored to the first surface by a polyacrylamide gel pad; a layer of polyvinylidene chloride affixed to the first surface of the slide by a layer of double-sided adhesive, wherein the adhesive layer is deposited on the first surface of the slide and encloses an area thereupon; a first and second port extending through the slide from the first surface to the second surface thereof each having a first opening and a second opening, wherein the first opening of each port is provided within the area on the first surface of the slide bounded by the adhesive and covered by the flexible layer and the second opening of each port is provided on the second surface of the slide, and wherein each port is in the shape of a truncated cone having a small-diameter end and a large diameter end, and wherein the small diameter end is the first opening and the large diameter end is the second opening; a layer of foil tape positioned over the second opening of each port; a layer of a polyethylene glycol positioned between the first surface of the slide and the layer of polyvinylidene chloride; a reflective layer positioned between the array and the first substrate surface; a layer of parylene positioned between the reflective layer and the and the layer of polyvinylidene chloride; and a resistive heater.

In a further aspect, the invention provides an apparatus for performing biological reactions, comprising a substrate having a first surface and a second surface, a multiplicity of biomolecules positioned on the first surface of the substrate, a flexible layer affixed to the first surface of the substrate by an adhesive layer, wherein the adhesive layer is deposited on the first surface of the substrate and encloses an area thereupon, and wherein a reaction volume is enclosed between the flexible layer and the first substrate surface in the area defined by the adhesive layer. There are first and second ports extending through the flexible layer and the adhesive layer into the volume enclosed between the flexible layer and the first substrate surface in the area defined by the adhesive layer.

In an additional aspect, each of the multiplicity of biomolecules is attached to an anchoring structure such as a gel pad of a polymeric material such as polyacrylamide, either as discrete sites or as a continuous layer.

In a further aspect, the apparatus comprises a label layer affixed to the flexible layer, wherein the first and second ports extend through the label layer and the flexible layer. The label layer can comprise an adhesive surface and a non-adhesive surface, and wherein the label layer is affixed to the flexible layer using the adhesive surface. The label layer can comprise a window corresponding in size and position to the area bounded by the adhesive layer, and wherein the window allows visual inspection of the flexible layer and the volume enclosed between the flexible layer and the first substrate surface in the area defined by the adhesive layer. Optionally, a reflective layer positioned between the array and the first substrate surface is provided, and/or a resistive heater.

In an additional aspect, the invention provides methods of using the apparatus to detect target analytes.

### BRIEF DESCRIPTION OF THE DRAWINGS

Presently preferred embodiments of the invention are described with reference to the following drawings.

FIGS. 1A-1D are views of a preferred embodiment of the present invention illustrating the preparation of a chamber for reaction. FIG. 1A is a cross-sectional view of the apparatus illustrating a hybridization chamber prefilled with a water-soluble compound in thermal contact with a heating element. FIG. 1B is a cross sectional view of the apparatus illustrating the mixing of the water-soluble compound and the biological sample fluid. FIG. 1C is a cross sectional view of the apparatus illustrating a chamber filled with the sample fluid/water-soluble compound mixture, wherein the first and second ports are covered with a seal. FIG. 1D is a top plan view of the apparatus illustrating the pattern of adhesive defining the individual areas containing the arrays of oligonucleotide probes.

FIG. 2 is an exploded cross-sectional view of a chamber showing the array of gel pads of a preferred embodiment of the invention.

FIG. 3 is an exploded perspective view of the array of biomolecular probes showing the positioning of the gel pads on the substrate of a preferred embodiment of the invention.

FIG. 4 is an exploded cross-sectional view of a port illustrating the conical shape of the port of a preferred embodiment of the invention.

FIG. 5 is a perspective view of the label layer, the flexible layer and the adhesive layer of a preferred embodiment of the invention.

FIG. 6 is a cross-sectional view of a stack of chambers according to a preferred embodiment.

FIGS. 7A-7E ac top views of the layers of an alternate preferred embodiment of the invention having inlet and outlet ports extending through the flexible layer. FIG. 7A is a view of the first adhesive layer, FIG. 7B is a view of the flexible layer, FIG. 7C is a view of the second adhesive layer, FIG. 7D is a view of the label layer, and FIG. 7E is a view of the layers of 7A to 7D as assembled.

FIGS. 8A-8B are detail views of the notches cut into the first adhesive layer and the label layer of a preferred embodiment of the invention having inlet and outlet ports extending through the flexible layer.

FIGS. 9A-9C are cross-sectional views of a preferred embodiment of the present invention illustrating the process of analyzing the array after completion of the reaction. FIG. 9A shows the apparatus upon completion of the reaction. FIG. 9B illustrates removal of the sample fluid from the chamber such that the flexible layer contacts the array. FIG. 9C illustrates use of a laser scanner to analyze the array.

FIGS. 10A-10C illustrate a handheld embodiment of the present invention. FIG. 10A is a side view of the hand held scanning system. FIG. 10B is a perspective view of a preferred embodiment comprising a hand-held scanning device illustrating the contact of the flexible layer with the carriage. FIG. 10C is a view of the handheld system illustrating the lid containing the display unit.

FIG. 11A-11E are cross-sectional views of the direct contact fiber optic scanner as shown in FIG. 10.

FIG. 12A-12C are alternate embodiments illustrating the apparatus coupled to a sample preparation chip. FIG. 12A illustrates an embodiment wherein the sample preparation chip is removably positioned against the second surface of the substrate. FIG. 12B illustrates an embodiment wherein the sample preparation chip is affixed to the second surface of the substrate. FIG. 12C illustrates an embodiment wherein the sample preparation chip is incorporated into the substrate.

FIG. 13 illustrates the assembly and use of a preferred embodiment of the present invention.

### DETAILED DESCRIPTION OF THE INVENTION

The present invention is directed to methods and compositions for performing high capacity biological reactions on a biochip comprising a substrate having an array of biological binding sites. The invention provides a reaction chamber, such as a hybridization chamber in the case where the biochip comprises nucleic acids. The hybridization chamber is formed with a substrate, a layer of adhesive and a flexible cover. The system utilizes ports, either in the substrate or in the flexible cover, to allow sample and/or reagent loading.

Accordingly, the present invention provides devices of the invention are used to detect target analytes in samples. By “target analyte” or “analyte” or grammatical equivalents herein is meant any molecule, compound or particle to be detected. As outlined below, target analytes preferably bind to binding ligands, as is more fully described above. As will be appreciated by those in the art, a large number of analytes may be detected using the present methods; basically, any target analyte for which a binding ligand, described herein, may be made may be detected using the methods of the invention.

Suitable analytes include organic and inorganic molecules, including biomolecules. In a preferred embodiment, the analyte may be an environmental pollutant (including pesticides, insecticides, toxins, etc.); a chemical (including solvents, polymers, organic materials, etc.); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc); whole cells (including procaryotic (such as pathogenic bacteria) and eukaryotic cells, including mammalian tumor cells); viruses (including retroviruses, herpesviruses, adenoviruses, lentiviruses, etc.); and spores; etc. Particularly preferred analytes are environmental pollutants; nucleic acids; proteins (including enzymes, antibodies, antigens, growth factors, cytokines, etc); therapeutic and abused drugs; cells; and viruses.

In a preferred embodiment, the target analyte is a nucleic acid. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Left. 4:395 (1994); Jeffs et al., J. Biomolecular NMR 34:17 (1994); Tetrahedron Left. 37:743 (1996)) and nonribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. Nucleic acid analogs also include “locked nucleic acids”. All of these references are hereby expressly incorporated by reference. These modifications of the ribose-phosphate backbone may be done to facilitate the addition of electron transfer moieties, or to increase the stability and half-life of such molecules in physiological environments.

As will be appreciated by those in the art, all of these nucleic acid analogs may find use in the present invention. In addition, mixtures of naturally occurring nucleic acids and analogs can be made; for example, at the site of conductive oligomer or electron transfer moiety attachment, an analog structure may be used. Alternatively, mixtures of different nucleic acid analogs, and mixtures of naturally occurring nucleic acids and analogs may be made.

As outlined herein, the nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occuring analog structures. Thus for example the individual units of a peptide nucleic acid, each containing a base, are referred to herein as nucleosides.

In a preferred embodiment, the present invention provides methods of detecting target nucleic acids. By “target nucleic acid” or “target sequence” or grammatical equivalents herein means a nucleic acid sequence on a single strand of nucleic acid. The target sequence may be a portion of a gene, a regulatory sequence, genomic DNA, cDNA, RNA including mRNA and rRNA, or others. It may be any length, with the understanding that longer sequences are more specific. In some embodiments, it may be desirable to fragment or cleave the sample nucleic acid into fragments of 100 to 10,000 basepairs, with fragments of roughly 500 basepairs being preferred in some embodiments. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others.

As is outlined more fully below, probes (including primers) are made to hybridize to target sequences to determine the presence or absence of the target sequence in a sample. Generally speaking, this term will be understood by those skilled in the art.

The target sequence may also be comprised of different target domains, which may be adjacent (i.e. contiguous) or separated. For example, when ligation chain reaction (LCR) techniques are used, a first primer may hybridize to a first target domain and a second primer may hybridize to a second target domain; either the domains are adjacent, or they may be separated by one or more nucleotides, coupled with the use of a polymerase and dNTPs, as is more fully outlined below. The terms “first” and “second” are not meant to confer an orientation of the sequences with respect to the 5′-3′ orientation of the target sequence. For example, assuming a 5′-3′ orientation of the complementary target sequence, the first target domain may be located either 5′ to the second domain, or 3′ to the second domain.

In a preferred embodiment, the target analyte is a protein. As will be appreciated by those in the art, there are a large number of possible proteinaceous target analytes that may be detected using the present invention. By “proteins” or grammatical equivalents herein is meant proteins, oligopeptides and peptides, derivatives and analogs, including proteins containing non-naturally occurring amino acids and amino acid analogs, and peptidomimetic structures. The side chains may be in either the (R) or the (S) configuration. In a preferred embodiment, the amino acids are in the (S) or L-configuration. As discussed below, when the protein is used as a binding ligand, it may be desirable to utilize protein analogs to retard degradation by sample contaminants.

Suitable protein target analytes include, but are not limited to, (1) immunoglobulins, particularly IgEs, IgGs and IgMs, and particularly therapeutically or diagnostically relevant antibodies, including but not limited to, for example, antibodies to human albumin, apolipoproteins (including apolipoprotein E), human chorionic gonadotropin, cortisol, α-fetoprotein, thyroxin, thyroid stimulating hormone (TSH), antithrombin, antibodies to pharmaceuticals (including antieptileptic drugs (phenytoin, primidone, carbariezepin, ethosuximide, valproic acid, and phenobarbitol), cardioactive drugs (digoxin, lidocaine, procainamide, and disopyramide), bronchodilators (theophylline), antibiotics (chloramphenicol, sulfonamides), antidepressants, immunosuppresants, abused drugs (amphetamine, methamphetamine, cannabinoids, cocaine and opiates) and antibodies to any number of viruses (including orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like), and bacteria (including a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi, Mycobacterum e.g. M. tuberculosis, M. leprae; Clostridium, e.g. C. botulinum, C. tetani, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lamblia Y. pestis, Pseudomonas, e.g. P. aeruginosa, P. putida; Chlamydia, e.g. C. trachomatis; Bordetella, e.g. B. pertussis; Treponema, e.g. T. palladium; and the like); (2) enzymes (and other proteins), including but not limited to, enzymes used as indicators of or treatment for heart disease, including creatine kinase, lactate dehydrogenase, aspartate amino transferase, troponin T, myoglobin, fibrinogen, cholesterol, triglycerides, thrombin, tissue plasminogen activator (tPA); pancreatic disease indicators including amylase, lipase, chymotrypsin and trypsin; liver function enzymes and proteins including cholinesterase, bilirubin, and alkaline phosphotase; aldolase, prostatic acid phosphatase, terminal deoxynucleotidyl transferase, and bacterial and viral enzymes such as HIV protease; (3) hormones and cytokines (many of which serve as ligands for cellular receptors) such as erythropoietin (EPO), thrombopoietin (TPO), the interleukins (including IL-1 through IL-17), insulin, insulin-like growth factors (including IGF-1 and -2), epidermal growth factor (EGF), transforming growth factors (including TGF-α and TGF-β), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cotrisol, estradiol, follicle stimulating hormone (FSH), thyroid-stimulating hormone (TSH), leutinzing hormone (LH), progeterone and testosterone; and (4) other proteins (including α-fetoprotein, carcinoembryonic antigen CEA, cancer markers, etc.).

In addition, any of the biomolecules for which antibodies may be detected may be detected directly as well; that is, detection of virus or bacterial cells, therapeutic and abused drugs, etc., may be done directly.

Suitable target analytes include carbohydrates, including but not limited to, markers for breast cancer (CA15-3, CA 549, CA 27.29), mucin-like carcinoma associated antigen (MCA), ovarian cancer (CA125), pancreatic cancer (DE-PAN-2), prostate cancer (PSA), CEA, and colorectal and pancreatic cancer (CA 19, CA 50, CA242).

Suitable target analytes include metal ions, particularly heavy and/or toxic metals, including but not limited to, aluminum, arsenic, cadmium, selenium, cobalt, copper, chromium, lead, silver and nickel.

These target analytes may be present in any number of different sample types, including, but not limited to, bodily fluids including blood, lymph, saliva, vaginal and anal secretions, urine, feces, perspiration and tears, and solid tissues, including liver, spleen, bone marrow, lung, muscle, brain, etc.

Accordingly, the present invention provides devices for the detection of target analytes comprising a solid substrate. The solid substrate can be made of a wide variety of materials and can be configured in a large number of ways, as is discussed herein and will be apparent to one of skill in the art. In addition, a single device may be comprises of more than one substrate; for example, there may be a “sample treatment” cassette that interfaces with a separate “detection” cassette; a raw sample is added to the sample treatment cassette and is manipulated to prepare the sample for detection, which is removed from the sample treatment cassette and added to the detection cassette. There may be an additional functional cassette into which the device fits; for example, a heating element which is placed in contact with the sample cassette to effect reactions such as PCR. In some cases, a portion of the substrate may be removable; for example, the sample cassette may have a detachable detection cassette, such that the entire sample cassette is not contacted with the detection apparatus. See for example U.S. Pat. No. 5,603,351, PCT US96/17116, and “MULTILAYERED MICROFLUIDIC DEVICES FOR ANALYTE REACTIONS” filed in the PCT Dec. 11, 2000 (no serial number received yet), hereby incorporated by reference.

The composition of the solid substrate will depend on a variety of factors, including the techniques used to create the device, the use of the device, the composition of the sample, the analyte to be detected, the size of the wells and microchannels, the presence or absence of electronic components, etc. Generally, the devices of the invention should be easily sterilizable as well.

In a preferred embodiment, the solid substrate can be made from a wide variety of materials including, but not limited to, silicon such as silicon wafers, silcon dioxide, silicon nitride, glass and fused silica, gallium arsenide, indium phosphide, aluminum, ceramics, polyimide, quartz, plastics, resins and polymers including polymethylmethacrylate, acrylics, polyethylene, polyethylene terepthalate, polycarbonate, polystyrene and other styrene copolymers, polypropylene, polytetrafluoroethylene, superalloys, zircaloy, steel, gold, silver, copper, tungsten, molybdeumn, tantalum, KOVAR, KEVLAR, KAPTON, MYLAR, brass, sapphire, etc. High quality glasses such as high melting borosilicate or fused silicas may be preferred for their UV transmission properties when any of the sample manipulation steps require light based technologies. In addition, as outlined herein, portions of the internal surfaces of the device may be coated with a variety of coatings as needed, to reduce non-specific binding, to allow the attachment of binding ligands, for biocompatibility, for flow resistance, etc.

In a preferred embodiment, the solid support comprises ceramic materials, such as are outlined in U.S. Ser. Nos. 09/235,081; 09/337,086; 09/464,490; 09/492,013; 09/466,325; 09/460,281; 09/460,283; 09/387,691; 09/438,600; 09/506,178; and 09/458,534; all of which are expressly incorporated by reference in their entirety. In this embodiment, the devices are made from layers of green-sheet that have been laminated and sintered together to form a substantially monolithic structure. Green-sheet is a composite material that includes inorganic particles of glass, glass-ceramic, ceramic, or mixtures thereof, dispersed in a polymer binder, and may also include additives such as plasticizers and dispersants. The green-sheet is preferably in the form of sheets that are 50 to 250 microns thick. The ceramic particles are typically metal oxides, such as aluminum oxide or zirconium oxide. An example of such a green-sheet that includes glass-ceramic particles is “AX951” that is sold by E. I. Du Pont de Nemours and Company. An example of a green-sheet that includes aluminum oxide particles is “Ferro Alumina” that is sold by Ferro Corp. The composition of the green-sheet may also be custom formulated to meet particular applications. The green-sheet layers are laminated together and then fired to form a substantially monolithic multilayered structure. The manufacturing, processing, and applications of ceramic green-sheets are described generally in Richard E. Mistler, “Tape Casting: The Basic Process for Meeting the Needs of the Electronics Industry,” Ceramic Bulletin, vol. 69, no. 6, pp. 1022-26 (1990), and in U.S. Pat. No. 3,991,029, which are incorporated herein by reference.

The method for fabricating devices (such as those depicted in FIGS. 27-30 as devices 100 and 200) begins with providing sheets of green-sheet that are preferably 50 to 250 microns thick. The sheets of green-sheet are cut to the desired size, typically 6 inches by 6 inches for conventional processing, although smaller or larger devices may be used as needed. Each green-sheet layer may then be textured using various techniques to form desired structures, such as vias, channels, or cavities, in the finished multilayered structure.

Various techniques may be used to texture a green-sheet layer. For example, portions of a green-sheet layer may be punched out to form vias or channels. This operation may be accomplished using conventional multilayer ceramic punches, such as the Pacific Trinetics Corp. Model APS-8718 Automated Punch System. Instead of punching out part of the material, features, such as channels and wells may be embossed into the surface of the green-sheet by pressing the green-sheet against an embossing plate that has a negative image of the desired structure. Texturing may also be accomplished by laser tooling with a laser via system, such as the Pacific Trinetics LVS-3012.

Next, a wide variety of materials may be applied, preferably in the form of thick-film pastes, to each textured green-sheet layer. For example, electrically conductive pathways may be provided by depositing metal-containing thick-film pastes onto the green-sheet layers. Thick-film pastes typically include the desired material, which may be either a metal or a dielectric, in the form of a powder dispersed in an organic vehicle, and the pastes are designed to have the viscosity appropriate for the desired deposition technique, such as screen-printing. The organic vehicle may include resins, solvents, surfactants, and flow-control agents. The thick-film paste may also include a small amount of a flux, such as a glass frit, to facilitate sintering. Thick-film technology is further described in J. D. Provance, “Performance Review of Thick Film Materials,” Insulation/Circuits (April, 1977) and in Morton L. Topfer, Thick Film Microelectronics, Fabrication, Design, and Applications (1977), pp. 41-59, which are incorporated herein by reference.

The porosity of the resulting thick-film can be adjusted by adjusting the amount of organic vehicle present in the thick-film paste. Specifically, the porosity of the thick-film can be increased by increased the percentage of organic vehicle in the thick-film paste. Similarly, the porosity of a green-sheet layer can be increased by increasing the proportion of organic binder. Another way of increasing porosity in thick-films and green-sheet layers is to disperse within the organic vehicle, or the organic binder, another organic phase that is not soluble in the organic vehicle. Polymer microspheres can be used advantageously for this purpose.

To add electrically conductive pathways, the thick film pastes typically include metal particles, such as silver, platinum, palladium, gold, copper, tungsten, nickel, tin, or alloys thereof. Silver pastes are preferred. Examples of suitable silver pastes are silver conductor composition numbers 7025 and 7713 sold by E. I. Du Pont de Nemours and Company.

The thick-film pastes are preferably applied to a green-sheet layer by screen-printing. In the screen-printing process, the thick-film paste is forced through a patterned silk screen so as to be deposited onto the green-sheet layer in a corresponding pattern. Typically, the silk screen pattern is created photographically by exposure to a mask. In this way, conductive traces may be applied to a surface of a green-sheet layer. Vias present in the green-sheet layer may also be filled with thick-film pastes. If filled with thick-filled pastes containing electrically conductive materials, the vias can serve to provide electrical connections between layers.

After the desired structures are formed in each layer of green-sheet, preferably a layer of adhesive is applied to either surface of the green-sheet. Preferably, the adhesive is a room-temperature adhesive. Such room-temperature adhesives have glass transition temperatures below room temperature, i.e., below about 20° C., so that they can bind substrates together at room temperature. Moreover, rather than undergoing a chemical change or chemically reacting with or dissolving components of the substrates, such room-temperature adhesives bind substrates together by penetrating into the surfaces of the substrates. Sometimes such room-temperature adhesives are referred to as “pressure-sensitive adhesives.” Suitable room-temperature adhesives are typically supplied as water-based emulsions and are available from Rohm and Haas, Inc. and from Air Products, Inc. For example, a material sold by Air Products, Inc. as “Flexcryl 1653” has been found to work well.

The room-temperature adhesive may be applied to the green-sheet by conventional coating techniques. To facilitate coating, it is often desirable to dilute the supplied pressure-sensitive adhesive in water, depending on the coating technique used and on the viscosity and solids loading of the starting material. After coating, the room-temperature adhesive is allowed to dry. The dried thickness of the film of room-temperature adhesive is preferably in the range of 1 to 10 microns, and the thickness should be uniform over the entire surface of the green-sheet. Film thicknesses that exceed 15 microns are undesirable. With such thick films of adhesive voiding or delamination can occur during firing, due to the large quantity of organic material that must be removed. Films that are less than about 0.5 microns thick when dried are too thin because they provide insufficient adhesion between the layers.

From among conventional coating techniques, spin-coating and spraying are the preferred methods. If spin-coating is used, it is preferable to add 1 gram of deionized water for every 10 grams of “Flexcryl 1653.” If spraying is used, a higher dilution level is preferred to facilitate ease of spraying. Additionally, when room-temperature adhesive is sprayed on, it is preferable to hold the green-sheet at an elevated temperature, e.g., about 60 to 70° C., so that the material dries nearly instantaneously as it is deposited onto the green-sheet. The instantaneous drying results in a more uniform and homogeneous film of adhesive.

After the room-temperature adhesive has been applied to the green-sheet layers, the layers are stacked together to form a multilayered green-sheet structure. Preferably, the layers are stacked in an alignment die, so as to maintain the desired registration between the structures of each layer. When an alignment die is used, alignment holes must be added to each green-sheet layer. Typically, the stacking process alone is sufficient to bind the green-sheet layers together when a room-temperature adhesive is used. In other words, little or no pressure is required to bind the layers together. However, in order to effect a more secure binding of the layers, the layers are preferably laminated together after they are stacked.

The lamination process involves the application of pressure to the stacked layers. For example, in the conventional lamination process, a uniaxial pressure of about 1000 to 1500 psi is applied to the stacked green-sheet layers that is then followed by an application of an isostatic pressure of about 3000 to 5000 psi for about 10 to 15 minutes at an elevated temperature, such as 70° C. Adhesives do not need to be applied to bind the green-sheet layers together when the conventional lamination process is used.

However, pressures less than 2500 psi are preferable in order to achieve good control over the dimensions of such structures as internal or external cavities and channels. Even lower pressures are more desirable to allow the formation of larger structures, such as cavities and channels. For example, if a lamination pressure of 2500 psi is used, the size of well-formed internal cavities and channels is typically limited to no larger than roughly 20 microns. Accordingly, pressures less than 1000 psi are more preferred, as such pressures generally enable structures having sizes greater than about 100 microns to be formed with some measure of dimensional control. Pressures of less than 300 psi are even more preferred, as such pressures typically allow structures with sizes greater than 250 microns to be formed with some degree of dimensional control. Pressures less than 100 psi, which are referred to herein as “near-zero pressures,” are most preferred, because at such pressures few limits exist on the size of internal and external cavities and channels that can be formed in the multilayered structure.

The pressure is preferably applied in the lamination process by means of a uniaxial press.

Alternatively, pressures less than about 100 psi may be applied by hand.

As with semiconductor device fabrication, many devices may be present on each sheet.

Accordingly, after lamination the multilayered structure may be diced using conventional green-sheet dicing or sawing apparatus to separate the individual devices. The high level of peel and shear resistance provided by the room-temperature adhesive results in the occurrence of very little edge delamination during the dicing process. If some layers become separated around the edges after dicing, the layers may be easily re-laminated by applying pressure to the affected edges by hand, without adversely affecting the rest of the device.

The final processing step is firing to convert the laminated multilayered green-sheet structure from its “green” state to form the finished, substantially monolithic, multilayered structure. The firing process occurs in two important stages as the temperature is raised. The first important stage is the binder burnout stage that occurs in the temperature range of about 250 to 500° C., during which the other organic materials, such as the binder in the green-sheet layers and the organic components in any applied thick-film pastes, are removed from the structure.

In the next important stage, the sintering stage, which occurs at a higher temperature, the inorganic particles sinter together so that the multilayered structure is densified and becomes substantially monolithic. The sintering temperature used depends on the nature of the inorganic particles present in the green-sheet. For many types of ceramics, appropriate sintering temperatures range from about 950 to about 1600° C., depending on the material. For example, for green-sheet containing aluminum oxide, sintering temperatures between 1400 and 1600° C. are typical. Other ceramic materials, such as silicon nitride, aluminum nitride, and silicon carbide, require higher sintering temperatures, namely 1700 to 2200° C. For green-sheet with glass-ceramic particles, a sintering temperature in the range of 750 to 950° C. is typical. Glass particles generally require sintering temperatures in the range of only about 350 to 700° C. Finally, metal particles may require sintering temperatures anywhere from 550 to 1700° C., depending on the metal.

Typically, the devices are fired for a period of about 4 hours to about 12 hours or more, depending on the material used. Generally, the firing should be of a sufficient duration so as to remove the organic materials from the structure and to completely sinter the inorganic particles. In particular, polymers are present as a binder in the green-sheet and in the room-temperature adhesive. The firing should be of sufficient temperature and duration to decompose these polymers and to allow for their removal from the multilayered structure.

Typically, the multilayered structure undergoes a reduction in volume during the firing process. During the binder burnout phase, a small volume reduction of about 0.5 to 1.5% is normally observed. At higher temperatures, during the sintering stage, a further volume reduction of about 14 to 17% is typically observed.

The volume change due to firing, on the other hand, can be controlled. In particular, to match volume changes in two materials, such as green-sheet and thick-film paste, one should match: (1) the particle sizes; and (2) the percentage of organic components, such as binders, which are removed during the firing process. Additionally, volume changes need not be matched exactly, but any mismatch will typically result in internal stresses in the device. But symmetrical processing, placing the identical material or structure on opposite sides of the device can, to some extent, compensate for shrinkage mismatched materials. Too great a mismatch in either sintering temperatures or volume changes may result in defects in or failure of some or all of the device. For example, the device may separate into its individual layers, or it may become warped or distorted.

As noted above, preferably any dissimilar materials added to the green-sheet layers are co-fired with them. Such dissimilar materials could be added as thick-film pastes or as other green-sheet layers, or added later in the fabrication process, after sintering. The benefit of co-firing is that the added materials are sintered to the green-sheet layers and become integral to the substantially monolithic microfluidic device. However, to be co-fireable, the added materials should have sintering temperatures and volume changes due to firing that are matched with those of the green-sheet layers. Sintering temperatures are largely material-dependent, so that matching sintering temperatures simply requires proper selection of materials. For example, although silver is the preferred metal for providing electrically conductive pathways, if the green-sheet layers contain alumina particles, which require a sintering temperature in the range of 1400 to 1600° C., some other metal, such as platinum, must be used due to the relatively low melting point of silver (961° C.).

Alternatively, the addition of other substrates or joining of two post-sintered pieces can be done using any variety of adhesive techniques, including those outlined herein. For example, two “halves” of a device can be glued or fused together. For example, a particular detection platform, reagent mixture such as a hydrogel or biological components that are not stable at high temperature can be sandwiched in between the two halves. Alternatively, ceramic devices comprising open channels or wells can be made, additional substrates or materials placed into the devices, and then they may be sealed with other materials.

A particularly preferred substrate is glass, such as a microscope slide.

In a preferred embodiment, the solid substrate is configured for handling a single sample that may contain a plurality of target analytes. That is, a single sample is added to the device and the sample may either be aliquoted for parallel processing for detection of the analytes or the sample may be processed serially, with individual targets being detected in a serial fashion. In addition, samples may be removed periodically or from different locations for in line sampling.

In a preferred embodiment, the solid substrate is configured for handling multiple samples, each of which may contain one or more target analytes. In general, in this embodiment, each sample is handled individually; that is, the manipulations and analyses are done in parallel, with preferably no contact or contamination between them. Alternatively, there may be some steps in common; for example, it may be desirable to process different samples separately but detect all of the target analytes on a single detection platform.

In addition, it should be understood that while most of the discussion herein is directed to the use of generally planar substrates with microchannels and wells, other geometries can be used as well. For example, two or more planar substrates can be stacked to produce a three dimensional device, that can contain microchannels flowing within one plane or between planes; similarly, wells may span two or more substrates to allow for larger sample volumes. Thus for example, both sides of a substrate can be etched to contain microchannels; see for example U.S. Pat. Nos. 5,603,351 and 5,681,484, both of which are hereby incorporated by reference.

The biochip substrates of the invention have capture binding ligands attached in array formats. By “array” or “biochip” herein is meant a plurality of capture binding ligands, preferably nucleic acids, in an array format; the size of the array will depend on the composition and end use of the array. Most of the discussion herein is directed to the use of nucleic acid arrays with attached capture probes, but this is not meant to limit the scope of the invention, as other types of capture binding ligands (proteins, etc.), can be used.

Nucleic acids arrays are known in the art, and can be classified in a number of ways; both ordered arrays (e.g. the ability to resolve chemistries at discrete sites), and random arrays are included. Ordered arrays include, but are not limited to, those made using photolithography techniques (Affymetrix GeneChip™), spotting techniques (Synteni and others), printing techniques (Hewlett Packard and Rosetta), three dimensional “gel pad” arrays, etc. The size of the array can vary; with arrays containing from about 2 different capture probes to many millions can be made, with very large arrays being possible. Generally, the array will comprise from two to as many as 100,000, with from about 400 to about 1000 being the most preferred, and about 10,000 being especially preferred. Arrays can also be classifed as “addressable”, which means that the individual elements of the array have precisely defined x and y coordinates, so that a given array element can be pinpointed.

The invention is advantageously used for performing assays using biochips 18. Biochips, as used in the art, encompass substrates containing arrays or microarrays, preferably ordered arrays and most preferably ordered, addressable arrays, of biological molecules that comprise one member of a biological binding pair. Typically, such arrays are oligonucleotide arrays comprising a nucleotide sequence that is complementary to at least one sequence expected to be present in a biological sample. Alternatively, peptides or other small molecules can be arrayed in such biochips for performing immunological analysis (wherein the arrayed molecules are antigens) or assaying biological receptors (wherein the arrayed molecules are ligands, agonists or antagonists of said receptors).

One useful feature of biochips is the manner in which the arrayed biomolecules are attached to the surface of the biochip. Conventionally such procedures involve multiple reaction steps, often requiring chemical modification of the solid support itself. Even in embodiments comprising absorption matrices, such as hydrogels, present on a solid support, chemical modification of the gel polymer is necessary to provide a chemical functionality capable forming a covalent bond with the biomolecule. The efficiency of the attachment chemistry and strength of the chemical bonds formed are critical to the fabrication and ultimate performance of the microarray.

Polymeric hydrogels and gel pads are used as binding layers to adhere to surfaces biological molecules including, but not limited to, proteins, peptides, oligonucleotides, polynucleotides, and larger nucleic acid fragments. The oligonucleotide probes may be bound to the surface of a continuous layer of the hydrogel, or to an array of gel pads. The gel pads comprising biochips for use with the apparatus of the present invention are conveniently produced as thin sheets or slabs, typically by depositing a solution of acrylamide monomer, a crosslinker such methylene bisacrylamide, and a catalyst such as N,N,N′,N′-tetramethylethylendiamine (TEMED) and an initiator such as ammonium persulfate for chemical polymerization, or 2,2-dimethoxy-2-phenyl-acetophone (DMPAP) for photopolymerization, in between two glass surfaces (e.g., glass plates or microscope slides) using a spacer to obtain the desired thickness of the polymeric gel. Generally, the acrylamide monomer and crosslinker are prepared in one solution of about 4-5% acrylamide (having an acrylamide/bisacrylamide ratio of 19/1) in water/glycerol, with a nominal amount of initiator added. The solution is polymerized and crosslinked either by ultraviolet (UV) radiation (e.g., 254 nm for at least about 15 minutes, or other appropriate UV conditions, collectively termed “photopolymerization”), or by thermal initiation at elevated temperature (e.g., typically at about 40° C.). Following polymerization and crosslinking, the top glass slide is removed from the surface to uncover the gel. The pore size (and hence the “sieving properties”) of the gel is controlled by changing the amount of crosslinker and the percent solids in the monomer solution. The pore size also can be controlled by changing the polymerization temperature.

In the fabrication of polyacrylamide embodiments of the polymeric hydrogel arrays of the invention (i.e., patterned gels) used as binding layers for biological molecules, the acrylamide solution typically is imaged through a mask during the UV polymerization/crosslinking step. The top glass slide is removed after polymerization, and the unpolymerized monomer is washed away (developed) with water, leaving a fine feature pattern of polyacrylamide hydrogel, which is used to produce the crosslinked polyacrylamide hydrogel pads. Further, in an application of lithographic techniques known in the semiconductor industry, light can be applied to discrete locations on the surface of a polyacrylamide hydrogel to activate these specified regions for the attachment of an oligonucleotide, an antibody, an antigen, a hormone, hormone receptor, a ligand or a polysaccharide on the surface (e.g., a polyacrylamide hydrogel surface) of a solid support (see, for example, International Application, Publication No. WO 91/07087, incorporated by reference).

For hydrogel-based arrays using polyacrylamide, biomolecules (such as oligonucleotides) are covalently attached by forming an amide, ester or disulfide bond between the biomolecule and a derivatized polymer comprising the cognate chemical group. Covalent attachment of the biomolecule to the polymer is usually performed after polymerization and chemical cross-linking of the polymer is completed.

Alternatively, oligonucleotides bearing 5′-terminal acrylamide modifications can be used that efficiently copolymerize with acrylamide monomers to form DNA-containing polyacrylamide copolymers (Rehman et al., 1999, Nucleic Acids Research 27: 649-655). Using this approach, stable probe-containing layers can be fabricated on supports (e.g., microtiter plates and silanized glass) having exposed acrylic groups. This approach has made available the commercially marketed “Acrydite™” capture probes (available from Mosaic Technologies, Boston, Mass.). The Acrydite moiety is a phosporamidite that contains an ethylene group capable of free-radical copolymerization with acrylamide, and which can be used in standard DNA synthesizers to introduce copolymerizable groups at the 5′ terminus of any oligonucleotide probe.

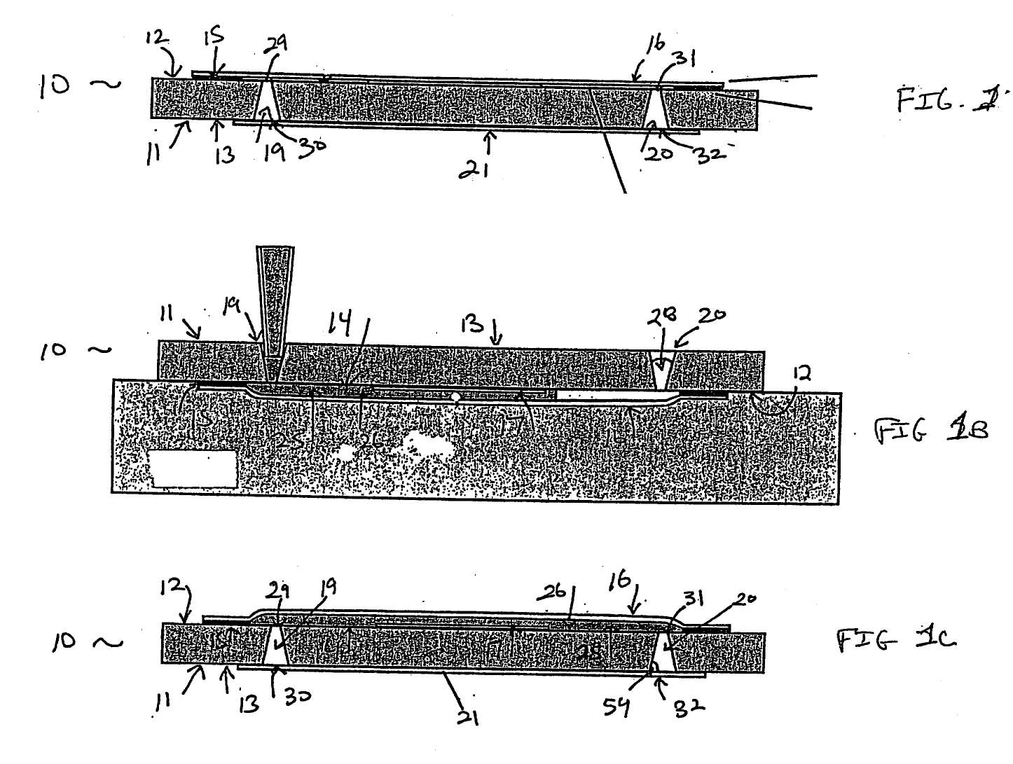

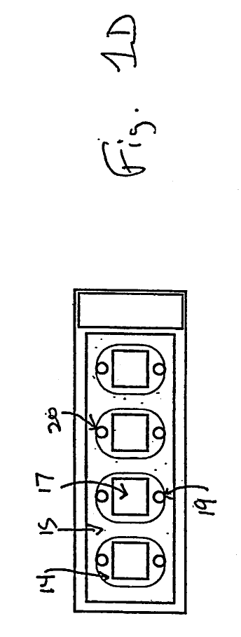

With reference to the illustration provided in FIG. 1, the invention provides a hybridization chamber 10 comprising a biochip, which comprises a substrate 11 having a first surface 12 and a second surface 13 opposite thereto, and a flexible layer 16 affixed to the first substrate surface 12 by an adhesive layer 15. On the first surface 12 is an area 14 bounded by adhesive layer 15 an completely covered by flexible layer 16. Flexible layer 16, adhesive layer 15, and first substrate surface 12 further define a volume 25. The ratio of volume 25 to area 14 is preferably from about 0.025 mL/mm² to about 0.25 mL/mm², more preferably from about 0.1 mL/mm² to about 0.25 mL/mm², and most preferably from about 0.1 mL/mm² to about 0.2 mL/mm².

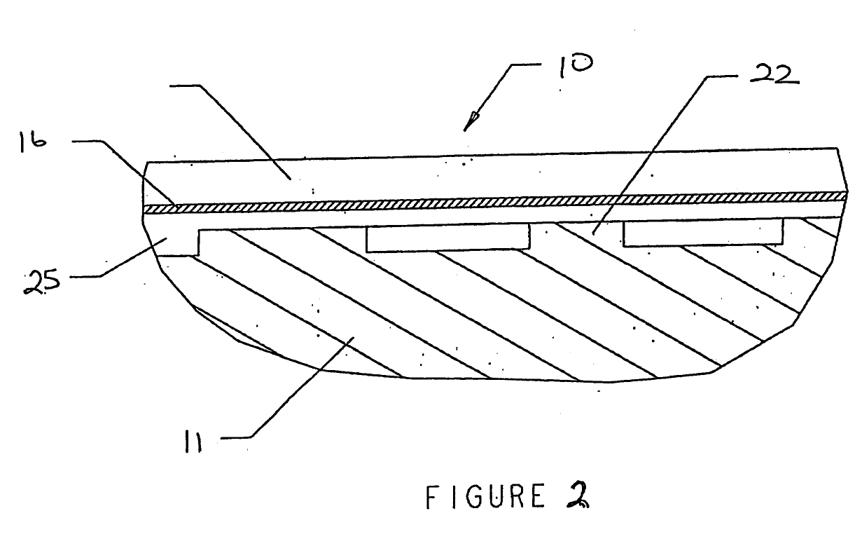

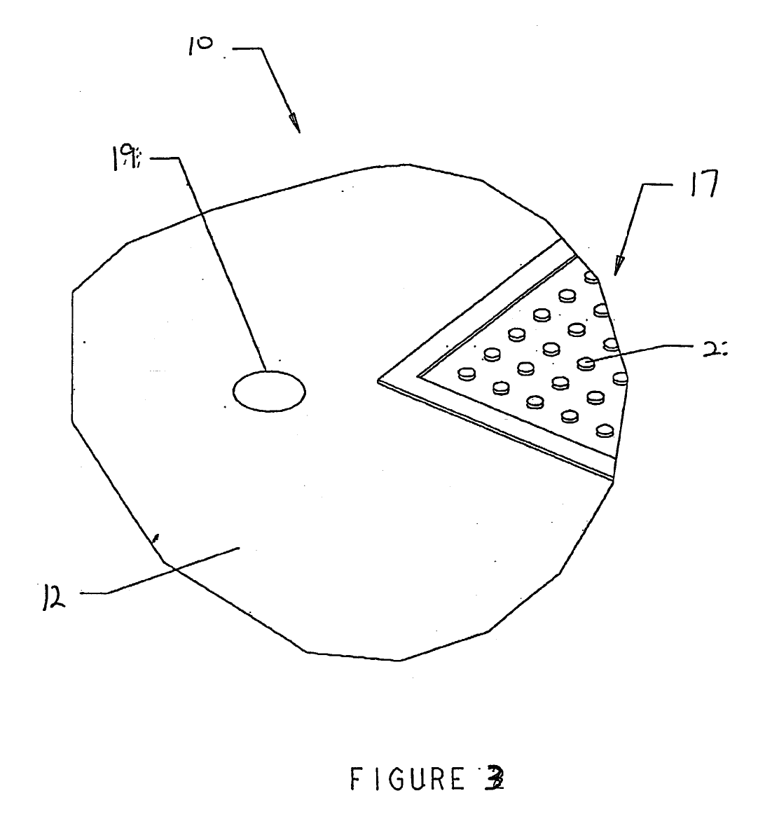

As shown in FIG. 3, between flexible layer 16 and first substrate surface 12 in area 14 is positioned a multiplicity of biomolecules. In a preferred embodiment, the multiplicity of biomolecules comprises an array 17 of biomolecules, which is preferably affixed to first substrate surface 12. Array 17 preferably further comprises gel pads 22. In an alternate preferred embodiment, array 17 is deposited on a continuous layer of polyacrylamide. FIG. 2 provides an exploded cross-sectional view of a portion of array 17 illustrating the gel pads 22. Each gel structure 22 is preferably cylindrical, most preferably having about a 113 micron diameter and about a 25 micron thickness. The distance between each site within each array 17 is most preferably about 300 microns.

A layer of a water-soluble compound 28 having a melting point of about 30 to about 60° C., more preferably of about 35 to about 50° C., and most preferably of about 35 to about 45° C. is deposited in volume 25 bounded by first substrate surface 12, flexible layer 16, and adhesive layer 15. Preferably, the water-soluble compound is biocompatible, does not stick to flexible layer 16, and serves to prevent mechanical damage to gel pads 22. This compound can comprise any number of materials, with polymers such as glycol polymers, dextrans, sugars and other carbohydrates being preferred. In a preferred embodiment, the compound is polyethylene glycol, most preferably polyethylene glycol 600. The compound 28 is deposited so that the entire volume 25, with the exception of that portion of volume 25 occupied by array 17, comprises compound 28.

Array 17 can be positioned on surface 12 by providing markings, most preferably holes or pits in surface 12, that act as fiducials or reference points on surface 12 for accurate placement of array 17. The presence of said fiducials is particularly advantageous in embodiments comprising a multiplicity of arrays 17 in one or a multiplicity of areas 14 on surface 12, wherein accurate placement of said arrays is required for proper spacing and orientation of the arrays on the hybridization chamber.

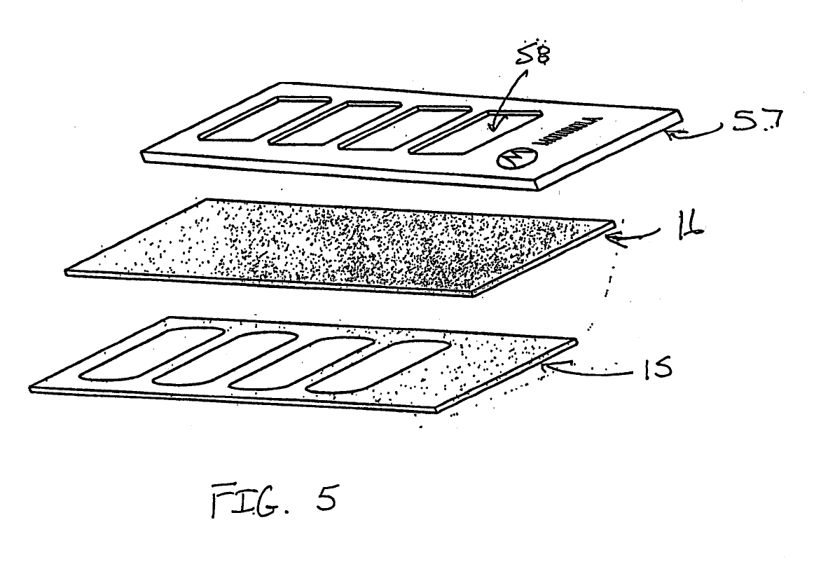

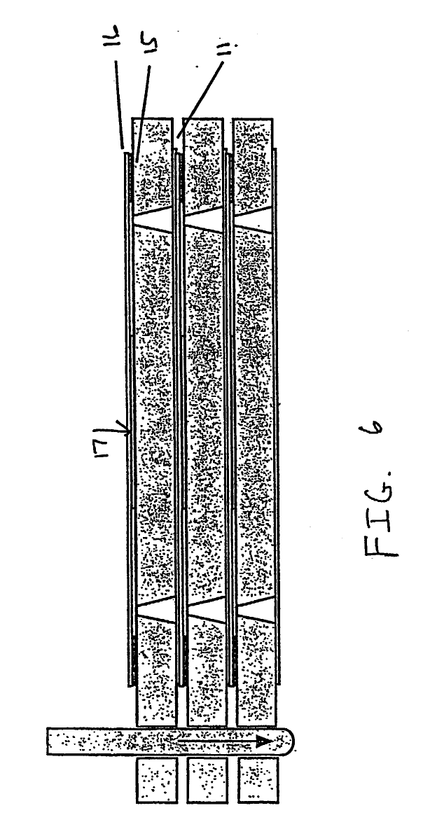

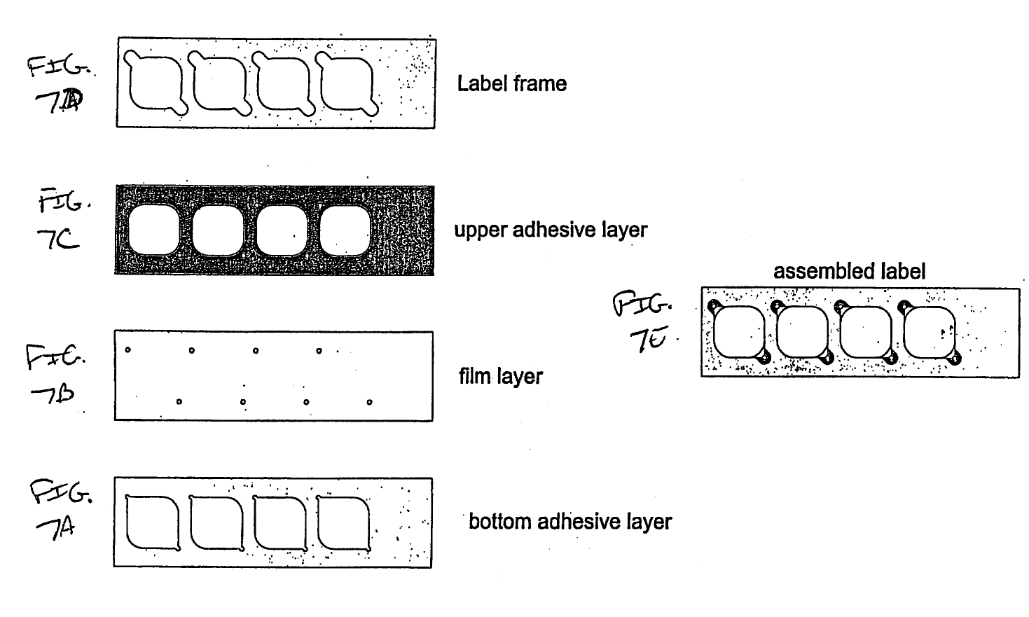

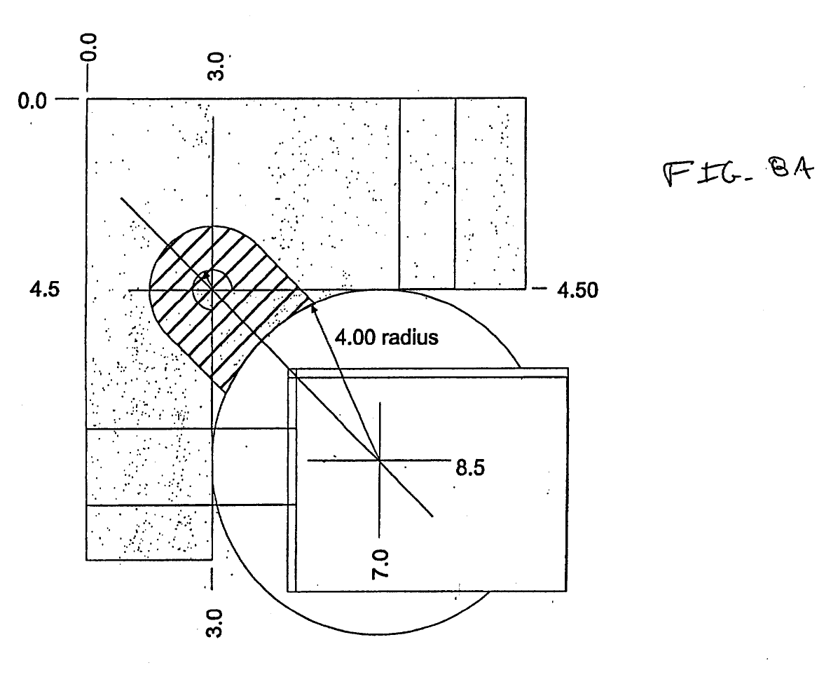

In preferred embodiments, a first and second port 19 and 20 extend through flexible layer 16, as shown in FIG. 7. The first port 19 serves as an input port and is positioned in flexible layer 16 so that the first opening 29 is provided within the area 14 bounded by adhesive layer 15 on first surface 12. Second port 20 serves as an outlet port and is positioned in flexible layer 16 so that the first opening 31 opens within area 14 bounded by the adhesive layer 15 on the first surface 12. The openings of ports 19 and 20 may be covered with a removable and replaceable cover 21. In preferred embodiments, replaceable cover 21 is a stopper, a gasket, or tape, most preferably a foil tape. In these embodiments, one or more first notches 70 are cut into the first adhesive layer 15 such that the first notches 70 are in direct communication with the area 14 on first substrate surface 12 bounded by the first adhesive layer 15. Second notches 72 are cut into the flexible layer 16 in positions corresponding to the size and position of first notches 70 in adhesive layer 15, thus forming one or more ports. In a particularly preferred embodiment, a ring of adhesive 74 is deposited around the perimeter of each second notch 72, such that the inner perimeter of adhesive ring 74 is coextensive with the outer perimeter of second notch 72. Preferably, first and second notches 70 and 72 are circular in shape, and have a diameter that is equal to the inner diameter of adhesive ring 74. Preferably the inner diameter and outer diameter of adhesive ring 74 are selected to form a tight seal with the tip end of a pipette. In an alternate preferred embodiment, a second layer of adhesive 76 is deposited on the portions of flexible layer 16 not covering the area 14 on first substrate surface 12 and not defining first and second ports 19 and 20. In this embodiment, the apparatus further comprises a label layer 57 that is die cut in the same manner as the first adhesive layer 15 to form windows 58 that correspond in location to areas 14 on first substrate surface 12, and which is applied to second adhesive layer 76. In this embodiment, one or more third notches 78 are cut into second adhesive layer 76, such that third notches 78 correspond in shape, size, and position to first and second notches 70 and 72. Fourth notches 80, having a shape and position corresponding to first, second and third notches 70, 72 and 78, are cut into label layer 57. The diameter of fourth notches 80 is preferably greater than the diameter of first, second and third notches 70, 72 and 78, such that after the apparatus is assembled a portion of second adhesive layer 76 is exposed by fourth notch 80. Preferably the exposed portion of second adhesive layer 76 corresponds to the shape and size of a pipette tip.

In alternative embodiments of the apparatus, first and second ports 19 and 20 extend through substrate 11, rather than through flexible layer 16. Illustrative embodiments are described in co-owned and co-pending U.S. application Ser. No. 09/464,490, incorporated by reference herein. In preferred embodiments of the apparatus, area 14 on first substrate surface 12 is square or rectangular with two rounded edges at diagonally opposite corners of are 14 and two 90 degree angles at the remaining two diagonally opposite corners of area 14. Preferably, when first and second ports 19 and 20 extend through flexible layer 16, first notches 70 in first adhesive layer 15 are cut at the sharp edges of area 14, as shown in FIG. 7. These embodiments are particularly preferred as they comprise geometries that eliminate comers and therefore are useful in the prevention of bubble formation in area 14.

Substrate 11 is fabricated from any solid supporting substance, including but not limited to plastics, metals, ceramics, and glasses. Most preferably, substrate 11 is made from silicon or glass (for accuracy and stiffness), molded plastics (which reduce cost of manufacture and thermal inertia), or ceramics (for the incorporation of microfluidic elements including integrated heating elements). Most preferably, the substrate is glass.

Adhesive layer 15 is prepared using an adhesive suitable for forming a water-tight bond between substrate 11 and flexible layer 16, including, but not limited to, high temperature acrylics, rubber-based adhesives, and silicone-based adhesives. The shape of adhesive layer 15 is configured to contain array 17. Adhesive layer 15 can be deposited on first substrate surface 12 in a pattern to produce area 14 in any desired shape, and is most preferably deposited to define an ellipsoid area 14. Adhesive layer 15 can be deposited using inkjet printing or offset printing methods, or by die cutting the desired shapes from a sheet of adhesive material. In addition, a substantial portion of first surface 12 can be covered with adhesive and portions of the substrate that are not desired to retain adhesive properties can be hardened preferentially, for example, by ultraviolet curing. In these embodiments, portions retaining adhesive properties can be defined using a mask and thereby retain adhesive properties necessary to affix flexible layer 16 to surface 12. In embodiments using the die cut adhesive material, the adhesive material is preferably a doublesided adhesive tape, and more preferably a double sided adhesive tape having no carrier. Adhesive layer 15 is most preferably set down in a layer between 1 and 100 mm thick, more preferably between 25 and 50 mm thick, and most preferably about 50 mm thick.

Flexible layer 16 is made of any flexible solid substance, including but not limited to plastics, including polypropylene, polyethylene, and polyvinylidene chloride (sold commercially as Saran® wrap) plastics, rubbers, including silicone rubbers, high temperature polyesters, and porous Teflon®. Flexible layer 16 is preferably biocompatible and preferably has low permeability in order to prevent evaporation of water from the volume contained between the flexible layer and the substrate. Flexible layer 16 also preferably is optically clear and should be able to withstand temperatures of between 50 and 95° C. for a period of between 8 and 12 hours without shrinkage. In a preferred embodiment, the flexible layer is a gas permeable membrane. Flexible layer 16 preferably covers an area of from about 5 mm² to about 1400 mm², more preferably from about 5 mm² to about 600 mm², and most preferably from about 100 mm² to about 600 mm².

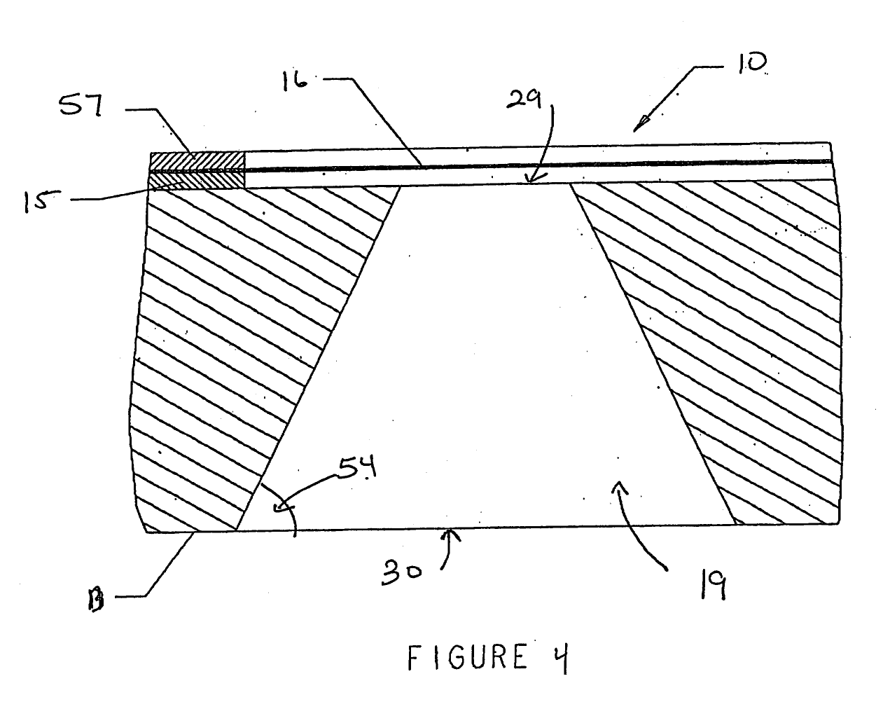

In preferred embodiments, as shown in FIG. 5, the invention further comprises a label layer 57 that is die cut in the same manner as the adhesive to form windows 58 that correspond in location to areas 14 on first substrate surface 12. The label layer is preferably a thick film having a layer of adhesive, and most preferably is an Avery laser label. The label layer is applied to the outer surface of the flexible layer, preferably by vacuum lamination.

Array 17 contained in area 14 on first substrate surface 12 is covered with a water-soluble compound 28, which protects and seals the biochip prior to use and prevents degradation or other damage to the array. Any water-soluble compound 28 having a melting point of about 30° C. to about 60° C., more preferably of about 35° C. to about 50° C., and most preferably of about 35° C. to about 45° C. is advantageously used in filling volume 25 between array 17 and flexible layer 16. Preferably, the compound is polyethylene glycol, most preferably polyethylene glycol 600. It is a particularly preferred feature of hybridization chamber 10 of the invention that water-soluble compound 28 fills the entirety of the volume 25 and more preferably also fills at least a portion of input port 19. This prevents formation of air bubbles in volume 25 because compound 28 is first melted, then carefully mixed with hybridization fluid 26 within volume 25 using a roller 40 without producing air bubbles in hybridization fluid 26. The lack of air bubbles in volume 25 minimizes artifactual signals detected by a scanner 36 or a light pipe 37.

Ports and holes can be produced in substrate 11 by diamond drilling in glass embodiments of substrate 11 or by stamping or molding in plastic embodiments thereof, or using ceramics formulation technology outlined herein. This facilitates standardization of the hybridization chamber dimensions, for example, by producing substrates where the first and second ports 19 and 20 are produced in a single operation. Both the substrate 11 and the removable cover 21 can be set down as strips or large sheets, and can be rolled to avoid trapping air bubbles. Flexible layer 16 can be applied by vacuum lamination to avoid trapping air, or can be deposited by spinning or flowing liquid plastic over substrate 11 after treatment with adhesive 15 and water-soluble compound 28, followed by curing the flexible layer in place. Individual hybridization chambers 10 can be produced in stacks using, for example, a diamond saw as shown in FIG. 6.

FIG. 6 illustrates a preferred arrangement for manufacturing hybridization chamber 10, wherein alternating layers of flexible layer 16, adhesive layer 15, uncut substrate 11, and removable cover 21 are laid down, and hybridization chambers are produced by cuffing the stacked layers, for example, with a diamond saw or any appropriate manufacturing tool. The sealed volumes 25 protect the arrays 17 from debris produced during the cutting process.

Alternative embodiments of the hybridization chamber 10 of the invention encompass a multiplicity of arrays 17 confined in a multiplicity of areas 14 underneath flexible layer 16, each area comprising an array 17 and being supplied with first port 19 and, optionally, second port 20. In these embodiments, adhesive layer 15 is deposited on first substrate surface 12 in a pattern that defines each of areas 14, and flexible layer 16 is applied to adhesive layer 15 to encompass areas 14 on said surface.

In certain embodiments of the invention, hybridization chamber 10 is produced containing array 17 or a multiplicity of arrays 17 as disclosed herein, wherein the chamber is provided ready-to-use by the addition of hybridization fluid 26 comprising one or a multiplicity of target molecules. In alternative embodiments, hybridization chamber 10 is provided without array 17, and allows for insertion thereof by a user. In these embodiments, at least one edge of flexible layer 16 is not adhered to first substrate surface 12.

In the use of the hybridization chamber 10 of the invention, an amount of a hybridization fluid 26, most preferably comprising a biological sample containing a target nucleic acid, is added to the hybridization chamber through first port 19. Before application of the hybridization fluid 26 to the chamber, volume 25 is most preferably heated to a temperature greater than or equal to the melting temperature of water-soluble compound 28. When melted, hybridization fluid 26 can be added to the chamber and mixed with the water-soluble compound, as shown in FIG. 1B. Preferably, water-soluble compound 28 does not affect hybridization in the chamber. More preferably, the amount of compound 28 is chosen such that hybridization efficiency is improved when compound 28 is mixed with sample fluid 26.

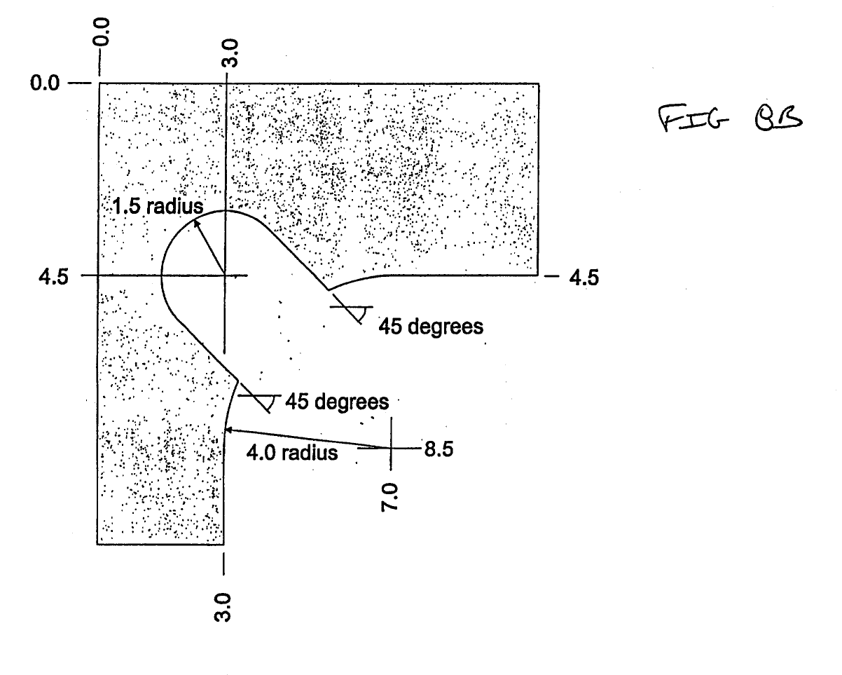

In embodiments of the chamber comprising first port 19 but not second port 20, the hybridization fluid is preferably introduced into the chamber after compound 28 is melted, and then the fluid is cycled into and out of the chamber using, most preferably, a pipette, until fluid 26 and compound 28 are fully mixed, and the hybridization fluid evenly distributed over the surface of array 17, or mixed into gel pads 22 comprising certain embodiments of said arrays. Alternatively, hybridization fluid 26 is evenly distributed over the surface of array 17, or mixed into gel pads 22 by physically manipulating flexible layer 16, as more fully described below. In these embodiments, hybridization fluid 26 is removed after hybridization is completed, as shown in FIG. 9, and array 17 is washed by the cycling a sufficient volume of a wash solution 27 into and out of the chamber, most preferably using a pipette. In embodiments of the chamber comprising both first port 19 and second port 20, the hybridization fluid is preferably introduced into the chamber after compound 28 is melted, and then the fluid is cycled into and out of the chamber using, most preferably, at least one pipette, until fluid 26 and compound 28 are fully mixed, and the hybridization fluid evenly distributed over the surface of array 17, or mixed into gel pads 22 comprising certain embodiments of said biochips. Hybridization is then performed by incubating the chamber for a time and at a temperature sufficient for hybridization to be accomplished. Hybridization fluid 26 is removed after hybridization has been completed using outlet port 20, and the biochip washed by the addition and cycling of a sufficient volume of a wash solution 27 into and out of the chamber, most preferably using a pipette. In these embodiments, the wash solution can also be continuously provided by addition through the input port and removal through the output port. In certain embodiments, the biochip containing the hybridized array is removed from the chamber for development or further manipulations as required. In preferred embodiments, the biochip containing the hybridized array is analyzed in situ as described below.

FIG. 1B illustrates an advantageous embodiment of hybridization chamber 10 of the invention, further comprising a heating element 33. Most advantageously, heating element 33 has a heating surface 34 adapted to the shape of hybridization chamber 10 to substantially cover the area 14 under flexible layer 16. Heating element 33 is any suitable heating means, including but not limited to resistance heaters, thermoelectric heaters, or microwave absorbing heaters.

The hybridization chamber 10 of the invention also advantageously comprises a thermocouple 35 or other temperature-sensing or measuring element to measure the temperature of hybridization fluid 26 or chamber 10. These temperature-sensing elements advantageously are coupled with heating element 33 to control hybridization fluid 26 and wash solution 27 temperature, and can be used to calibrate other elements, such as scanning devices 36 as described below that may be sensitive to temperature.

In certain embodiments of the invention, positive hybridization is detected visually, i.e., by the production of a dye or other material that reflects visible light at sites on biochip 18 where hybridization has occurred. In these embodiments, the dye or other material is most preferably produced enzymatically, for example, using a hybridization-specific immunological reagent such as an antibody linked to an enzyme that catalyzes the production of the dye. Visual inspection can be used to detect sites of positive hybridization. More preferably, the biochip containing the hybridized array is scanned using scanner 36 as disclosed more fully below.

Positive hybridization on biochip 18 most preferably is detected by fluorescence using labeled target molecules in a biological sample, or by including intercalating dyes in the hybridization fluid 26 that fluoresce when bound by a hybridized DNA duplex and illuminated by light at a particular wavelength. Suitable intercalating dyes include, but are not limited to, ethidium bromide, Hoechst DAPI, and Alexa Fluor dyes. Suitable fluorescence labels include, but are not limited to, fluorescein, rhodamine, propidium iodide, and Cy3 and Cy5 (Amersham), that can be incorporated into target molecules, for example, in vitro amplified fragments using labeled oligonucleotide primers.

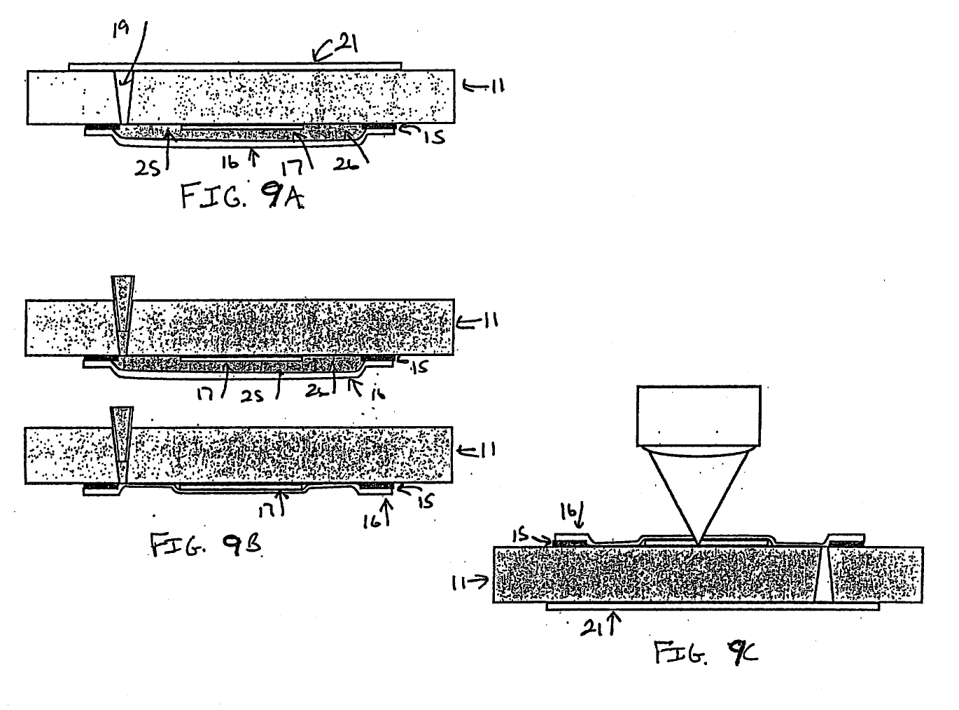

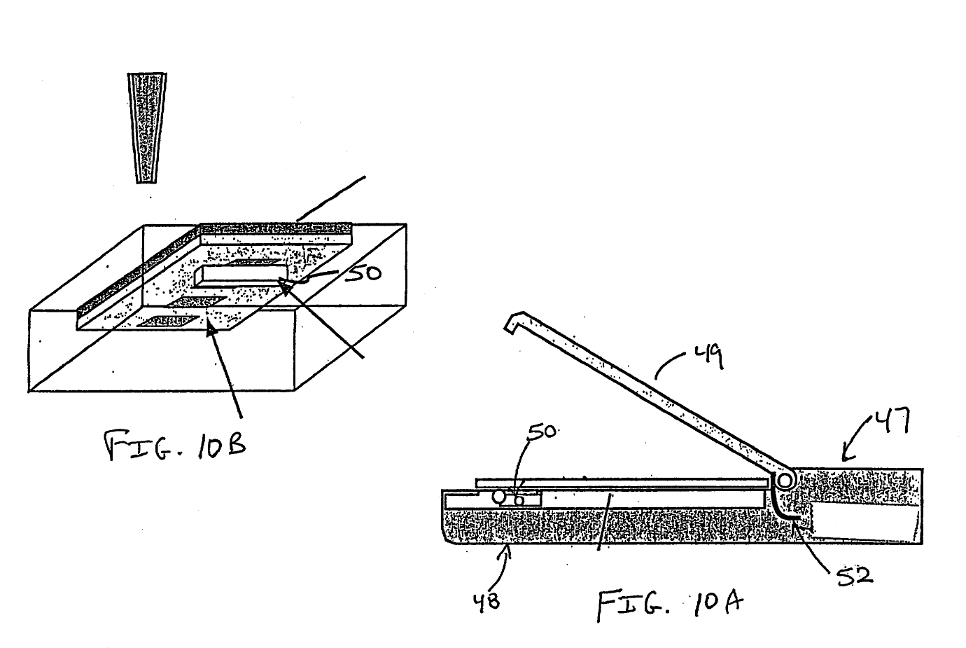

FIGS. 10A-10C illustrate an embodiment of the invention comprising a scanner 36, which is advantageously positioned over (or beneath) flexible layer 16 and moves from one end of area 14 to the opposite end, sequentially illuminating area 14 and array 17 positioned thereupon. Prior to analysis of the hybridized array, all fluid is removed from volume 25 such that flexible layer 16 is in contact with array 17. Scanner 36 then excites the fluorescent dye, preferably with short wavelength light, most preferably light with a wavelength between 250 nm and 600 nm. Scanner 36 then collects the emitted light from a specific area. The amount of light emitted is then used to determine the amount of fluorescent dye present in the area, and hence the amount of labeled target.

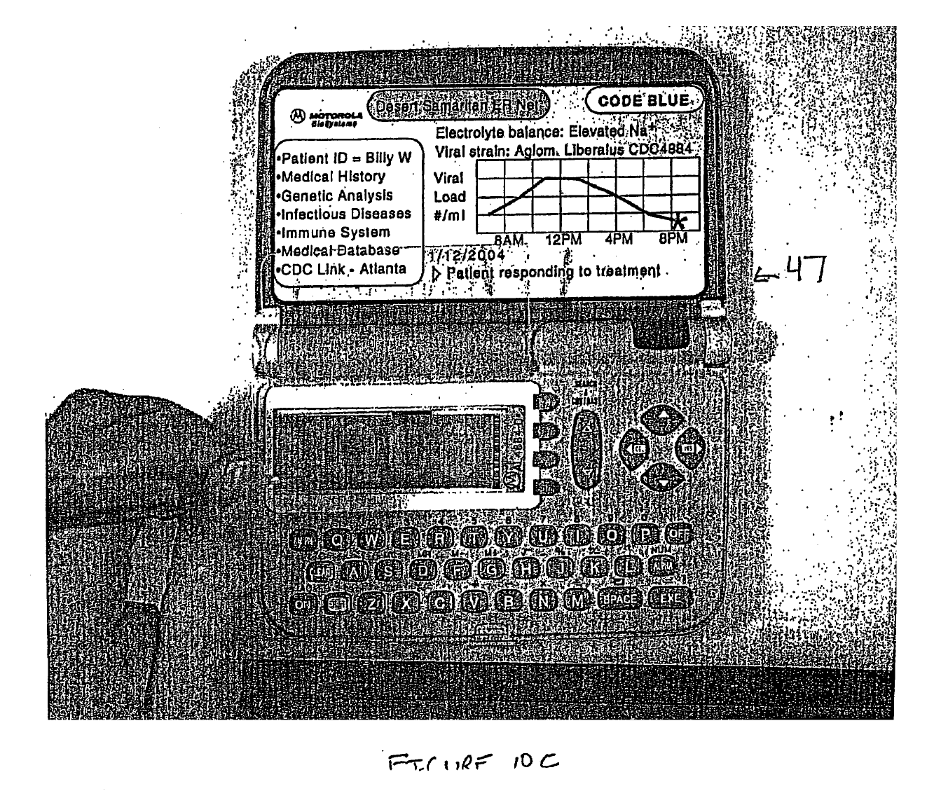

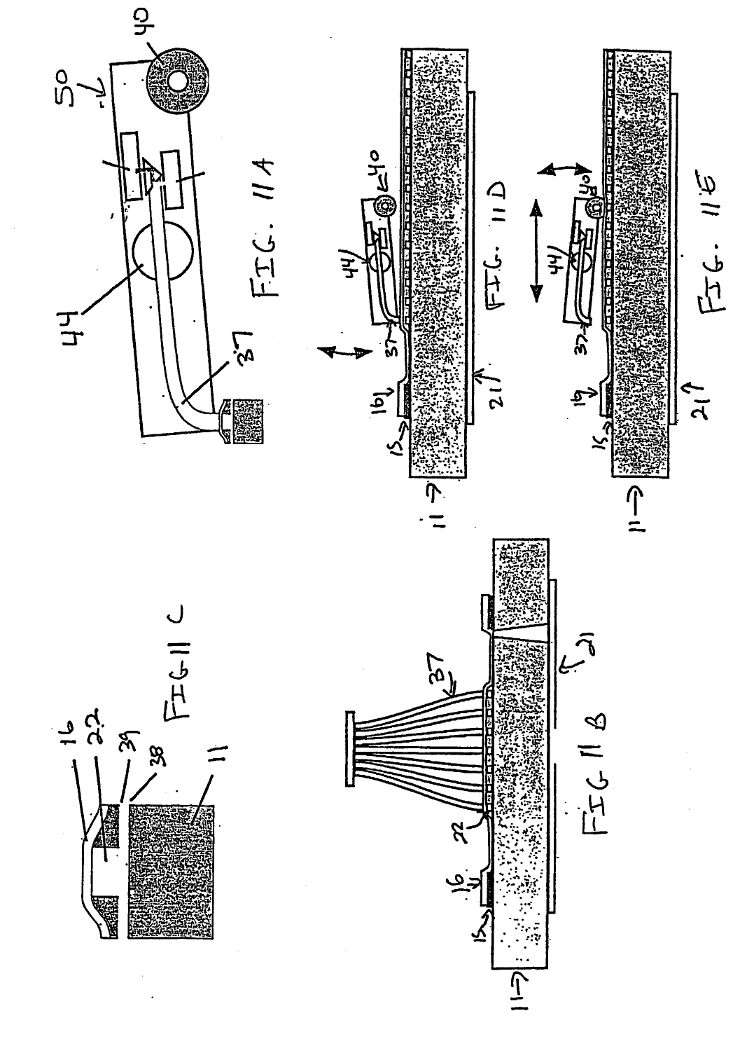

Particular embodiments of scanners and scanning devices 36 are shown in FIGS. 11A through 11E. It is a particularly advantageous feature of hybridization chamber 10 that flexible layer 16 is translucent to suitable wavelengths of light, including light in the ultraviolet and visible portion of the spectrum. An additional advantageous feature of hybridization chamber 10 is that flexible layer 16, which is very thin, is immediately adjacent to and in contact with biochip 18 after hybridization fluid 26 or wash fluid 27 is removed from the chamber. This combination of features reduces or eliminates free surface reflections, internal reflection of illumination from the scanner, and dispersion or scattering of illuminating light, thereby optimizing the amount of incident light that illuminates array 17. This arrangement is also more economical than in existing apparatus as it minimizes the need for highly polished, low scattering surfaces or complex or expensive lenses, and eliminates problems associated with focus and depth-of-field in more complex optical detectors.

In other embodiments, a light pipe 37 contacts the surface of flexible layer 16 that is immediately adjacent to and in contact with the surface of array 17, as shown in FIG. 11B. In these embodiments, both illuminating and emitted light are conveyed and collected by light pipe 37. The pipe is designed to be slightly flexible so as to adapt to the contoured surface of flexible layer 16. Light pipe 37 contacts flexible layer 16 that contacts array 17, thereby permitting contacts free of surface reflections even under circumstances where array 17 or light pipe 37 has localized variations in height. Advantageously, light pipe 37 has a larger surface area than array 17, so that the maximum amount of illuminating light is delivered to array 17, and the maximum amount of emitted light from array 17 is collected by light pipe 37. A further advantage of light pipe 37 is that it enables detection of bubbles formed in hybridization fluid 26 or wash buffer 27, which detection can be used as a signal for roller 40 to address flexible layer 16 to remove such bubbles. Removing bubbles in hybridization fluid 26 or wash buffer 27 reduces the frequency of non-specific binding and artifactual signals detected by scanner 36.

In additional embodiments of the invention, the area 14 defined by adhesive layer 15 further comprises a reflective layer 38 that substantially covers the entirety of the area 14 and is positioned between array 17 and the first substrate surface 12. In preferred embodiments, reflective layer 38 comprises aluminum, gold, silver, or platinum. In these embodiments, the amount of the light signal reflected or transmitted to the light-detecting portion of scanner 36 is increased up to four-fold. In further advantageous embodiments of the invention, reflective layer 38 is a metal film resistor or an RF induction heater. In these embodiments, reflective 38 layer can heat the slide without requiring additional heating elements 33. This is a particularly desirable feature in hand-held embodiments of the hybridization chamber 10 of the invention.

If required, a passivation later 39 can be applied on top of reflective layer 38. Preferably, passivation layer 39 is a layer of parylene a few microns thick that is applied by evaporation. The amount of illumination required, and hence the amount of power needed to operate scanner 36 is reduced in these embodiments, which are particularly suited to battery-operated embodiments such as hand-held devices to improve useful battery life. Furthermore, passivation layer 39 reduces artifactual signals in the light emission data by obscuring objects that it covers.

Hybridization chamber 10 is preferably supplied with a roller 40 in removable contact with flexible layer 16 and that can be moved longitudinally across areas 14 on first substrate surface 12. In preferred embodiments, the surface of roller 40 comprises a textured pattern 41, most preferably a spiral pattern, that permits the roller to efficiently mix hybridization fluid and wash solution across area 14 and array 17. One advantageous arrangement of roller 40 and hybridization chamber 10 is shown in FIG. 11E. As shown in the Figure, roller 40 can be advantageously connected to a movable arm 42 that can be positioned to place roller 40 in contact with flexible layer 16 when in a first position, and can be moved to a second position in which roller 40 is not in contact with flexible layer 16. Most preferably, movable arm 42 has a pivot point 44 and movement about said pivot point is preferably controlled by a solenoid. In addition to movement of roller 40 in contact with and away from hybridization chamber 10, either roller 40 or hybridization chamber 10, or both, are movable in a longitudinal direction to enable roller 40 to mix hybridization fluid 26 or wash solution 27 inside volume 25 bounded by flexible layer 16, adhesive layer 15, and first substrate surface 12 in area 14 containing array 17. In embodiments comprising a multiplicity of areas 14 containing a multiplicity of arrays 17, roller 40 is positioned to move longitudinally across each of the multiplicity of areas 14 to mix hybridization fluid 26 or wash solution 27 in each of the volumes 25 containing arrays 17.

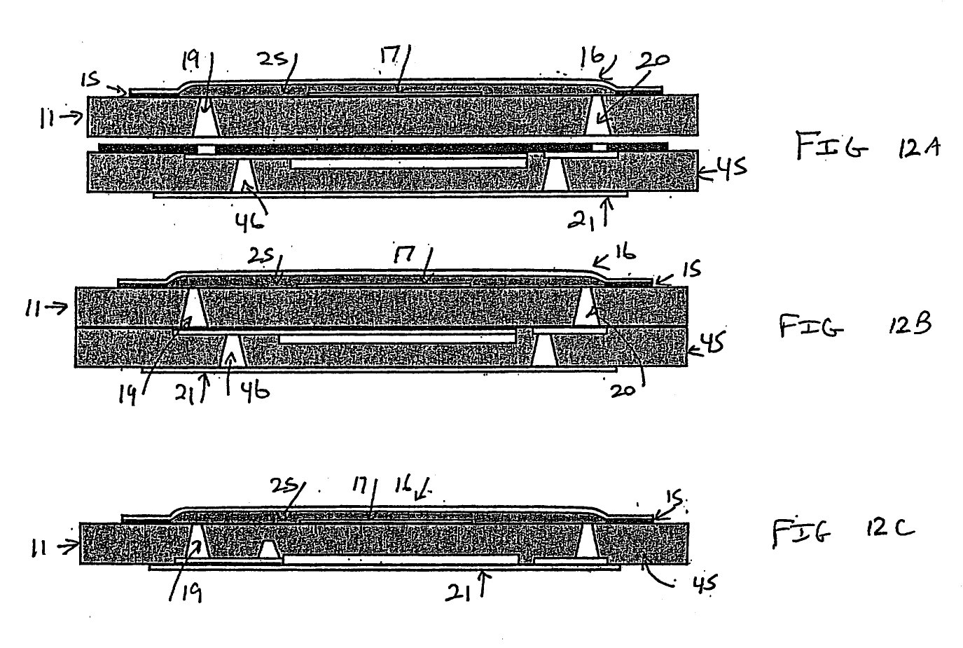

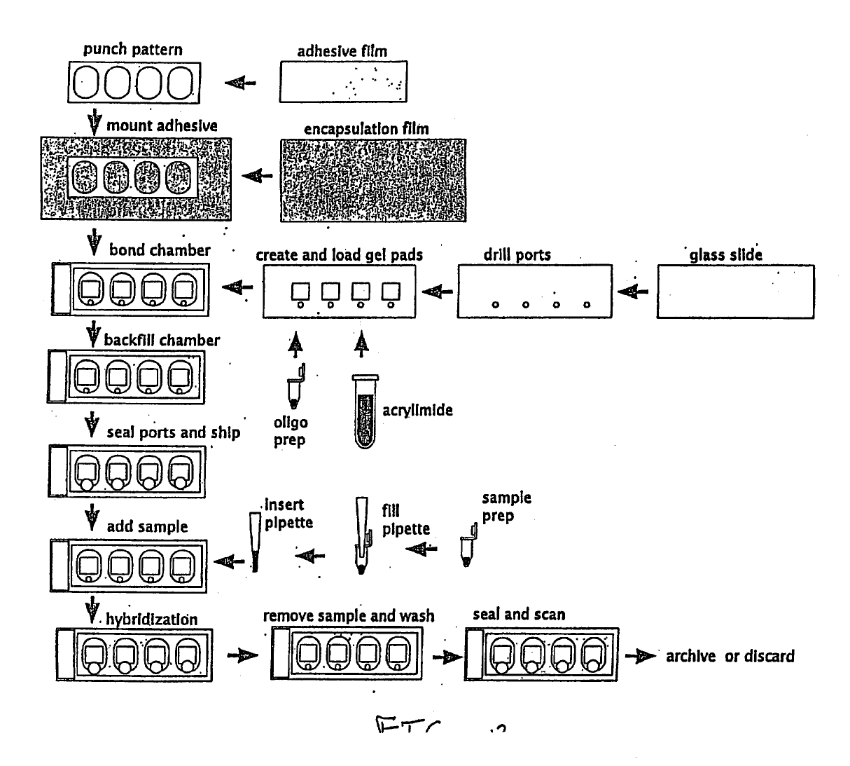

In additional embodiments, a sample preparation chip 45, comprising a port 46, as shown in FIGS. 12A through 12C, can be attached to hybridization chamber 10. Most preferably, port 46 in sample preparation chip 45 is aligned with first port 19 in hybridization chamber 10 to permit efficient transfer of sample to volume 25. Additional fiducial references can be used to accurately align hybridization chamber 10 and sample preparation chip 45. Since access to first port 19 is through second substrate surface 13, the array can be scanned without interference from the attached sample preparation chip. In alternative embodiments of the invention, sample preparation chip 45 may be bound to second substrate surface 13 (FIG. 12B) or formed as an integral part of substrate 11 (FIG. 12C).

A preferred embodiment of hybridization chamber 10 of the invention is a hand-held embodiment as shown in FIGS. 10A-10C, further comprising a scanner 36. In these embodiments, hand-held device 47 comprises a base 48, a lid 49 and a carriage 50 embodying roller 40, scanner 36, heating element 33 and thermocouple 35. Carriage 50 is illustrated in FIG. 11A. Device 47 comprises a compartment 51 for positioning hybridization chamber 10 in proximity to carriage 50. Carriage 50 is provided with moving means for moving roller 40, scanner 36 and heating element 33 relative to hybridization chamber 10 as required for operation as described above. Carriage 50 and lid 49 are arranged to permit a user to introduce and remove hybridization fluid 26 and wash solution 27 into the chamber through first port 19 and second port 20 as required. Alternatively, device 47 further comprises fluidic connections 52 to each of the first and second ports to provide for sample introduction and array washing after hybridization of the sample thereto. Device 47 is most preferably operated by battery, although AC adapters are also advantageously encompassed by the description of the device herein. In further preferred embodiments, lid 49 further comprises a display 56 for displaying the results of the analysis.

With respect to the methods of using the devices, there are a wide variety of methods that can be used. If required, the target sequence is prepared using known techniques. For example, the sample may be treated to lyse the cells, using known lysis buffers, sonication, electroporation, etc., with purification and amplification as outlined below occurring as needed, as will be appreciated by those in the art. In addition, the reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents which may be included in the assays. These include reagents like salts, buffers, neutral proteins, e.g. albumin, detergents, etc., which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target.

In addition, in most embodiments, double stranded target nucleic acids are denatured to render them single stranded so as to permit hybridization of the primers and other probes of the invention. A preferred embodiment utilizes a thermal step, generally by raising the temperature of the reaction to about 95° C., although pH changes and other techniques may also be used.

As outlined herein, the invention provides a number of capture probes that will hybridize to some portion, i.e. a domain, of the target sequence. Probes of the present invention are designed to be complementary to a target sequence (either the target sequence of the sample or to other probe sequences, for example for use in sandwich assays known in the art) such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions.

A variety of hybridization conditions may be used in the present invention, including high, moderate and low stringency conditions; see for example Maniatis et al., Molecular Cloning: A Laboratory Manual, 2d Edition, 1989, and Short Protocols in Molecular Biology, ed. Ausubel, et al, hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength and pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of helix destabilizing agents such as formamide. The hybridization conditions may also vary when a non-ionic backbone, i.e. PNA is used, as is known in the art. In addition, cross-linking agents may be added after target binding to cross-link, i.e. covalently attach, the two strands of the hybridization complex.

Thus, the assays are generally run under stringency conditions which allows formation of the hybridization complex only in the presence of target. Stringency can be controlled by altering a step parameter that is a thermodynamic variable, including, but not limited to, temperature, formamide concentration, salt concentration, chaotropic salt concentration, pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

As described herein, there are a number of possible detection techniques that can be utilized in the present invention. In a preferred embodiment, as outlined herein, optical label techniques are used. In these embodiments, a label such as an optical dye (e.g. a fluorochrome) is added to the assay complex comprising the target analyte and the capture binding ligand. In some embodiments, for example in the case of nucleic acids, the label can be added to the target, for example by incorporation during an amplification reaction such as PCR. For example, the fluorochromes or other labels such as biotin can be added to the PCR primers or to the dNTPs for enzymatic incorporation. Alternatively, intercalators can be used as described above.

Alternatively, preferred embodiments allow the use of electrical detection methods such as those outlined in U.S. Ser. Nos. 09/458,553; 09/458,501; 09/572,187; 09/495,992; 09/344,217; WO00/31148; 09/439,889; 09/438,209; 09/344,620; 09/478,727; PCTUS00/17422; WO 98/20162; WO 98/12430; WO 98/57158; WO 99/57317; WO 99/67425; PCT 00/19889; and WO 99/57319, all of which are expressly incorporated by reference in their entirety. These embodiments utilize arrays of microelectrodes on the substrate.

The Examples that follow are illustrative of specific embodiments of the invention and various uses thereof. They are set forth for explanatory purposes only, and are not to be taken as limiting the invention. All references cited herein are expressly incorporated by reference in their entirety.

### EXAMPLE 1

Assembly of a Hybridization Chamber

The process of assembling a chamber according to the present invention is illustrated in FIG. 13.

A die cutter was used to cut four ellipsoidal holes in a layer of 502FL ultra-clean laminating adhesive film (3M). A similar pattern was punched into an Avery laser label 5663 for use as a frame and label layer. Meanwhile, a sheet of polyvinylidene chloride film was stretched over a stainless steel frame and annealed for 30 minutes at 100° C. The Avery label was applied to one side of the polyvinylidine chloride film by vacuum laminating the label in a vacuum lamination press. A vacuum of 15 psi was applied for 30 seconds, and mechanical pressure of 15 psi was maintained for 1 minute after release of the vacuum. The adhesive was then applied to the opposite side of the polyvinylidene chloride film using the same process as for the label.

The adhesive coated film was then applied to a glass slide that had previously been prepared. The arrays of oligonucleotide probes and gel pads were positioned on the glass slide using standard methods. Ports were drilled into the slide using a diamond drill. A vacuum lamination press was used to affix the polyvinylidene chloride film to the slide. A vacuum of 15 psi was maintained for 1 minute, and then mechanical pressure of 15 psi was maintained for an additional minute.

The individual chambers were then filled with polyethylene glycol 600 using a 10 mL pipette tip. A layer of 3M 7350 polyester tape was then applied to the slide to seal off the ports.

### EXAMPLE 2

Assembly of a Top-loading Hybridization Chamber

A die cutter was used to cut four ellipsoidal holes in a layer of 502FL ultra-clean laminating adhesive film (3M). A similar pattern was punched into an Avery laser label 5663 for use as a frame and label layer. Meanwhile, a sheet of polyvinylidene chloride film was stretched over a stainless steel frame and annealed for 30 minutes at 100° C. The Avery label was applied to one side of the polyvinylidine chloride film by vacuum laminating the label in a vacuum lamination press. A vacuum of 15 psi was applied for 30 seconds, and mechanical pressure of 15 psi was maintained for 1 minute after release of the vacuum. The adhesive was then applied to the opposite side of the polyvinylidene chloride film using the same process as for the label.

The adhesive coated film was then applied to a glass slide that had previously been prepared. The arrays of oligonucleotide probes and gel- pads were positioned on the glass slide using standard methods. A vacuum lamination press was used to affix the polyvinylidene chloride film to the slide. A vacuum of 15 psi was maintained for 1 minute, and then mechanical pressure of 15 psi was maintained for an additional minute.

The individual chambers were then filled with polyethylene glycol 600 using a 10 mL pipette tip. A layer of 3M 7350 polyester tape was then applied to the slide to seal off the ports.

I claim:

1. An apparatus for performing biological reactions comprising:
a) a substrate having a first surface and a second surface;
b) an array of biomolecules positioned on said first surface;
c) a flexible layer affixed to said first surface by an adhesive layer to create a reaction volume; and
d) a port extended from said second surface to said reaction volume of said first surface.

2. An apparatus for performing biological reactions comprising:
a) a substrate having a first surface;
b) an array of biomolecules positioned on said first surface; and
c) a flexible layer affixed to said first surface by an adhesive layer to create a reaction volume, comprising at least a first port into said reaction volume.

3. An apparatus according to claim 1 or 2 wherein said substrate is glass, silicon, ceramic or plastic.

4. An apparatus according to claim 1, 2 or 3 wherein said biomolecules are nucleic acids.

5. An apparatus according to claim 1, 2, 3 or 4 wherein reaction volume comprises a water soluble compound.

6. An apparatus according to claim 1, 2, 3, 4 or 5 wherein said biomolecules are attached to said substrate using a gel pad.

7. An apparatus according to claim 1, 2, 3, 4, 5 or 6 further comprising a scanner.
